(19) United States
(12) Patent Application Publication
(10) Pub. No.: US 2025/0318595 A1
(43) Pub. Date: Oct. 16, 2025

(54) Sleep Cap Device

(71) Applicant: Martha Stewart, Blue Bell, PA (US)

(72) Inventor: Martha Stewart, Blue Bell, PA (US)

(21) Appl. No.: 19/057,096

(22) Filed: Feb. 19, 2025

Related U.S. Application Data
(60) Provisional application No. 63/634,027, filed on Apr. 15, 2024.

(51) Int. Cl. A42B 1/049; A61F 9/04

(52) U.S. Cl. CPC A42B 1/049; A61F 9/04

(57) ABSTRACT

A sleep cap device is disclosed which comprises a bonnet component that is configured in a round base shape of a traditional sleep cap or bonnet. The detachable sleep mask is secured to the edge of the bonnet component with a set of hook and loop fasteners. Further, the hook and loop fasteners allow the sleep mask to not wrap all the way around the head and also to be detached if necessary. Thus, the bonnet component sits on the head so that if attached, the sleep mask covers the eyes of the user. The detachable sleep mask could be a hot or cold sleep mask, as well. Further, the bonnet component can also include different types of attachment styles than hook and loop fasteners. An elastic band is included in the bonnet component to secure the bonnet component to the user's head.

### CROSS-REFERENCE TO RELATED APPLICATION

The present application claims priority to, and the benefit of, U.S. Provisional Application No. 63/634,027, which was filed on Apr. 15, 2024, and is incorporated herein by reference in its entirety.

### FIELD OF THE INVENTION

The present invention relates generally to the field of sleep cap devices. More specifically, the present invention relates to a sleep cap with a detachable sleep mask. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in sleep cap devices.

Generally, people may opt to use sleep bonnets and masks for a more restful night's sleep while preserving a hairstyle. Indentations on the face and hairstyle due to a traditional full strap sleep mask can be frustrating. Accordingly, it is cumbersome to wear a sleep mask and bonnet simultaneously, and people may feel stifled by having to tighten the eye mask over the bonnet. Further, not wearing a sleep mask can lead to delayed onset of sleep, headaches, and tired eyes if a person has trouble falling asleep quickly.

Accordingly, a sleep cap constructed in accordance with the invention that can promote better sleep and protect hairstyles is desirable. Further, it is often necessary for people to sleep in their hairstyles in order to have them functional the following day. This would require a sleep cap or bonnet over the hairstyle, so it still remains intact and stylish the following day. Accordingly, a sleep mask is usually used with the sleep cap as well to ensure maximum comfort and better sleep for the user.

Accordingly, there is a demand for an improved sleep cap device that allows users to comfortably sleep with a sleep cap and a sleep mask. More particularly, there is a demand for a sleep cap device that protects hairstyles from getting damaged or ruined overnight from a full strap sleep mask.

Therefore, there exists a long felt need in the art for a sleep cap device that provides users with an improved sleep cap or bonnet and a detachable sleep mask. There is also a long felt need in the art for a sleep cap device that allows users to keep their eyes covered with the mask and maintain a desired hairstyle via the bonnet. Further, there is a long felt need in the art for a sleep cap device that enables users to interchange the mask with hook and loop fastener attachments, featuring different types of masks such as a hot or cold type for headache or sinus relief. Moreover, there is a long felt need in the art for a device that ensures a hairstyle remains sleek and intact without developing indentations on the sides and back of the head due to a traditional sleep mask. Further, there is a long felt need in the art for a sleep cap device that includes numerous sizes, colors, and styles to accommodate all user needs and preferences. Finally, there is a long felt need in the art for a sleep cap device in which the design can also be applied to headbands, lace melting bands, sports headbands, and head wraps.

The subject matter disclosed and claimed herein, in one embodiment thereof, comprises a sleep cap device. The device is a modified sleep cap or bonnet with a detachable sleep mask. The sleep cap device comprises a bonnet component that is configured in a round base shape of a traditional sleep cap or bonnet. The detachable sleep mask is secured to the edge of the bonnet component with a set of hook and loop fasteners. Further, the hook and loop fasteners allow the sleep mask to not wrap all the way around the head and also to be detached if necessary. Thus, the bonnet component sits on the head so that if attached, the sleep mask covers the eyes of the user. The detachable sleep mask could be a hot or cold sleep mask, as well. Further, the bonnet component can also include different types of attachment styles than hook and loop fasteners. An elastic band is included in the bonnet component to secure the bonnet component to the user's head.

In this manner, the sleep cap device of the present invention accomplishes all of the forgoing objectives and provides users with an improved sleep cap or bonnet and detachable sleep mask. The device is a sleep cap with a sleep mask attached to it via hook and loop fasteners. The device can comprise a hot or cold sleep mask as well as a traditional one.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sleep cap device. The device is a nighttime garment set for promoting better sleep and protecting hairstyles. The sleep cap device comprises a bonnet component with a detachable sleep mask. Further, the detachable sleep mask is attached to the bonnet component via hook and loop fasteners placed at the edge of the bonnet component. Thus, the bonnet component is placed on the user's head so that, when attached, the sleep mask covers the eyes of the user. Further, the sleep mask could also be a hot or cold sleep mask rather than a traditional sleep mask.

In one embodiment, the sleep cap device is also suitable for travel and especially helpful for shift workers who need to sleep during the day. The detachable nature of the mask lends itself for fast removal which can be helpful when flying or needing to rise quickly such as when someone is knocking at your door etc.

In one embodiment, the sleep cap device comprises unisex options which include, female bonnets (which are sized small, medium, or large, short, mid-length, or long, depending upon hair length); scarves; melt bands; lock caps/scarves; headbands; traditional head wraps/scarves. Further, men's variations include, du-rag, do-rag, skull cap, silk cap, or knitted cap (which are all suitable for travel).

In one embodiment, the sleep cap device allows users to keep their eyes covered with a mask and still maintain a desired hairstyle via the bonnet. Further, the sleep mask is detachable so that a user is not required to use one. Accordingly, the sleep mask can be detached so that either a hot or cold sleep mask could be attached instead.

In one embodiment, the sleep cap device comprises a bonnet component that is configured in the shape of a traditional sleep bonnet to protect hairstyles and is suitable for sleep and travel. The bonnet component comprises an outer surface, an inner surface, and an elastic edge. However, the bonnet component can be any suitable shape and size as is known in the art, as long as the bonnet component is sized and shaped to fully cover a user's head/hairstyle.

In one embodiment, a securing means is added to the elastic edge of the bonnet component. This securing means is traditionally hook and loop fasteners, but can be any suitable securing means as is known in the art. This securing means allows the sleep mask to attach and detach from the bonnet component at will. The securing means being on the elastic edge of the bonnet component also assists in protecting the hairstyle of the user as the sleep mask is no longer secured to the user with an elastic strap around the entire hairstyle, but merely on the outer edge of the bonnet component. The sleep mask can be secured to any suitable position on the bonnet component as is known in the art.

In one embodiment, the sleep mask can be a gel sleep mask (which can be heated or cooled, as desired), as well, to help users who might require either temperature. This can be achieved by an attachment of a sleep mask having a gel material within it that can be heated or cooled, as desired, to the hook and loop fasteners on the elastic edge of the bonnet component. This gel sleep mask can be frozen to cool it down or heated to warm it up, thus achieving the temperature relief necessary to the user.

In one embodiment, the sleep mask can also be a weighted sleep mask. This weighted sleep mask would still attach to the hook and loop fasteners on the elastic edge of the bonnet component but would be heavier than a traditional sleep mask to help people with anxiety feel calmer because of an increase in pressure or weight.

In one embodiment, the sleep mask can be a blackout sleep mask. Further, the sleep masks can include an interchangeable set of a regular mask, hot/cold therapy mask, and a blackout mask, and could be packaged as a kit.

In one embodiment, the sleep mask can comprise separate gel inserts to make it a hot or cold mask.

In one embodiment, the sleep cap device comprises an optional ribbon around the elastic edge of the bonnet component. This ribbon covers the securing means after the sleep mask is either attached or detached to allow for a more comfortable sleep. The ribbon also then is tied together at the top of the user's head and can allow for more security so that the bonnet component stays on the user's head while sleeping. This ribbon would be sewn into the outer edge of the bonnet component so it is not a stand-alone attachment.

In one embodiment, the bonnet component and sleep mask are completely collapsible for storage. Specifically, the bonnet component and sleep mask can be folded down into a small area to fit in a bag or some other storage container. With a smaller and more compact storing arrangement, the device is easier to transport and keep inside the user's bedroom.

In one embodiment, the sleep cap device is manufactured from cotton, polyester, nylon, silk, satin, or other suitable materials as is known in the art, or a combination of such materials.

In yet another embodiment, the sleep cap device comprises a plurality of indicia.

In yet another embodiment, a method of protecting a hairstyle while sleeping is disclosed. The method includes the steps of providing a sleep cap device comprising a bonnet component with detachable sleep mask. The method also comprises stretching the elastic edge wide. Further, the method comprises placing the hairstyle inside the bonnet component. The method also comprises ensuring the elastic edge is secured around the user's head. Finally, the method comprises attaching the detachable sleep mask to the hook and loop fasteners on the elastic edge.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side perspective view of one embodiment of the sleep cap device of the present invention showing the securing means attaching the sleep mask to the bonnet in accordance with the disclosed architecture;

FIG. 2 illustrates a perspective view of one embodiment of the sleep cap device of the present invention showing the device in use in accordance with the disclosed architecture;

FIG. 3 illustrates a side perspective view of one embodiment of the sleep cap device of the present invention showing the different bonnets that can be used in accordance with the disclosed architecture;

FIG. 4 illustrates a side perspective view of one embodiment of the sleep cap device of the present invention showing the variety of bonnets and securing means in accordance with the disclosed architecture; and

FIG. 5 illustrates a flowchart showing the method of protecting a hairstyle while sleeping in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a sleep cap device that provides users with an improved sleep cap or bonnet and a detachable sleep mask. There is also a long felt need in the art for a sleep cap device that allows users to keep their eyes covered with the mask and maintain a desired hairstyle via the bonnet. Further, there is a long felt need in the art for a sleep cap device that enables users to interchange the mask with hook and loop fastener attachments, featuring different types of masks, such as a hot or cold type for headache or sinus relief. Moreover, there is a long felt need in the art for a device that ensures a hairstyle remains sleek and intact without developing indentations on the sides and back of the head due to a traditional sleep mask. Further, there is a long felt need in the art for a sleep cap device that includes numerous sizes, colors, and styles to accommodate all user needs and preferences. Finally, there is a long felt need in the art for a sleep cap device in which the design can also be applied to headbands, lace melting bands, sports headbands, and head wraps.

The present invention, in one exemplary embodiment, is a novel sleep cap device. The sleep cap device comprises a bonnet component that is configured in a round base shape of a traditional sleep cap or bonnet. The detachable sleep mask is secured to the edge of the bonnet component with a set of hook and loop fasteners. Further, the hook and loop fasteners allow the sleep mask to not wrap all the way around the head and also to be detached if necessary. Thus, the bonnet component sits on the head so that if attached, the sleep mask covers the eyes of the user. The detachable sleep mask could be a hot or cold sleep mask as well. Further, the bonnet component can also include different types of attachment styles than hook and loop fasteners. An elastic band is included in the bonnet component to secure the bonnet component to the user's head. The present invention also includes a novel method of providing a sleep cap device comprising a bonnet component with detachable sleep mask. The method also comprises stretching the elastic edge wide. Further, the method comprises placing the hairstyle inside the bonnet component. The method also comprises ensuring the elastic edge is secured around the user's head. Finally, the method comprises attaching the detachable sleep mask to the hook and loop fasteners on the elastic edge. The present invention will also be beneficial in a spa setting as it may be used as a spa head wrap or turban.

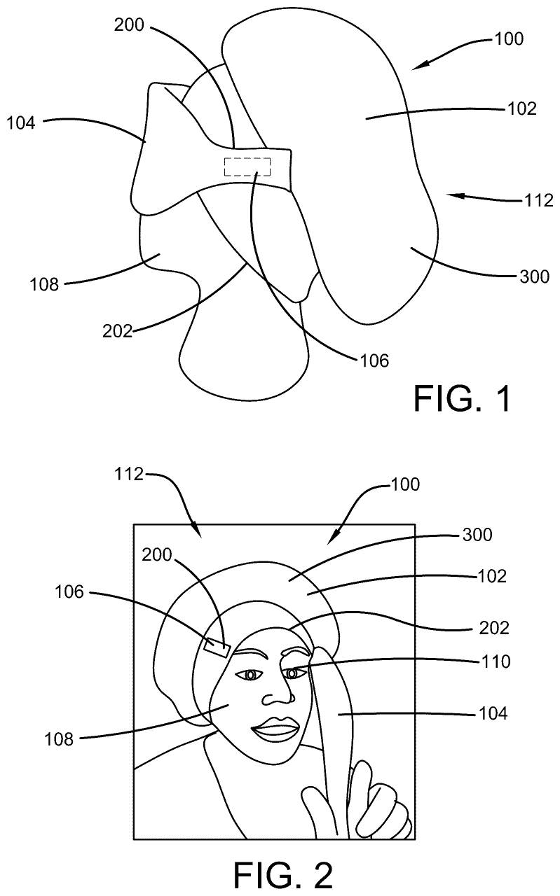

Referring initially to the drawings, FIG. 1 illustrates a perspective view of one embodiment of the sleep cap device 100 of the present invention. In the present embodiment, the sleep cap device 100 is a nighttime garment set for promoting better sleep and protecting hairstyles. The sleep cap device 100 comprises a bonnet component 102 with a detachable sleep mask 104. Further, the detachable sleep mask 104 is attached to the bonnet component 102 via hook and loop fasteners 106 placed at the edge of the bonnet component 102. Thus, the bonnet component 102 is placed on the user's head 108 so that, when attached, the detachable sleep mask 104 covers the eyes 110 of the user 112.

Generally, the sleep cap device 100 allows users 112 to keep their eyes 110 covered and still maintain a desired hairstyle via the bonnet component 102 and is suitable for sleep and travel. A traditional sleep mask would wrap around the user's head 108 and essentially flatten or otherwise ruin the intended hairstyle. Thus, the detachable sleep mask 104 allows for a strap that is wrapped around the user's head 108 to be unnecessary. The sleep cap device 100 is manufactured from cotton, polyester, nylon, silk, satin, or other suitable materials as is known in the art, or a combination of such materials.

As shown in FIG. 2, a securing means 200 is added to the elastic edge 202 of the bonnet component 102. This securing means 200 is traditionally hook and loop fasteners 106, but can be any suitable material as is known in the art. This securing means 200 allows the detachable sleep mask 104 to be detachable and can be placed on or off the device 100 depending on the will of the user 112. The securing means 200 being on the elastic edge 202 of the bonnet component 102 also assists in protecting the hairstyle of the user 112 as the detachable sleep mask 104 is no longer secured to the user 112 as previously mentioned with a strap around the user's head 108. The securing means 200 can be positioned on any suitable position of the bonnet component 102 as is known in the art, depending on the needs and/or wants of a user.

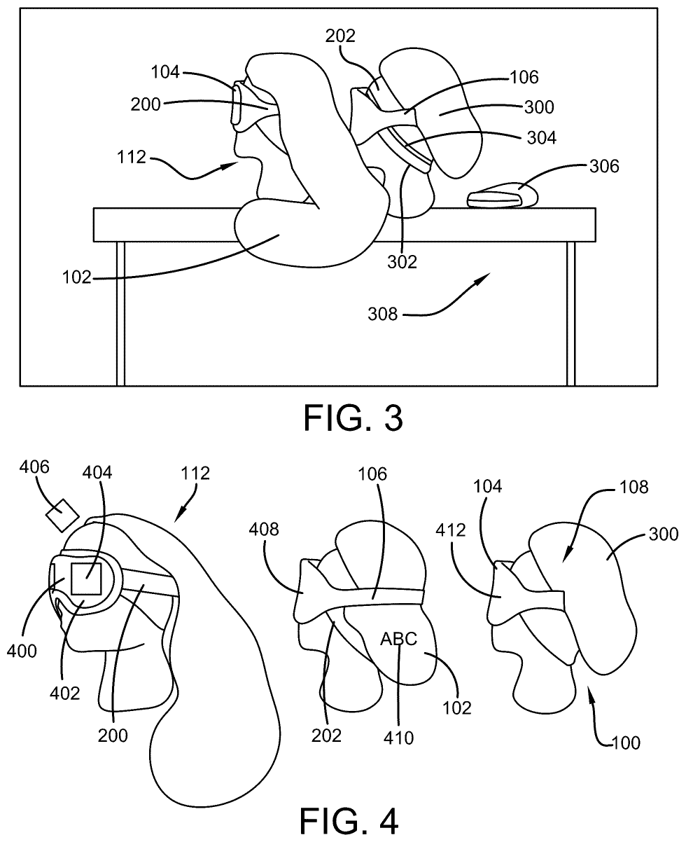

As shown in FIG. 3, the sleep cap device 100 comprises a bonnet component 102 that is configured in the shape of a traditional sleep bonnet which are normally used in the protection of hairstyles. The bonnet component 102 comprises an outer surface 300, an inner surface 302, and an elastic edge 202. However, the bonnet component 102 can be any suitable shape and size as is known in the art, as long as the bonnet component 102 is sized and shaped to fully cover a user's head 108 as well as comfortably protect their hairstyle.

In one embodiment, the sleep cap device 100 comprises unisex options which include, female bonnets (which are sized small, medium, or large, short, mid-length, or long, depending upon hair length); scarves; melt bands; lock caps/scarves; headbands; traditional head wraps/scarves. Further, men's variations include, du-rag, do-rag, skull cap, silk cap, or knitted cap (which are all suitable for travel).

Additionally, the sleep cap device 100 comprises an optional ribbon 304 around the elastic edge 202 of the bonnet component 102. This ribbon 304 covers the securing means 200 after the detachable sleep mask 104 is either attached or detached to allow for a more comfortable sleep. The ribbon 304 secures to the bonnet component 102 around the elastic edge 202 of the bonnet component 102 which also allows the bonnet component 102 to be secure to the user's head 108. In another embodiment, this ribbon 304 could be sewn into the elastic edge 202 of the bonnet component 102 so it is not a stand-alone attachment.

In yet another embodiment, the bonnet component 102 and detachable sleep mask 104 are completely collapsible for storage. Specifically, the bonnet component 102 and detachable sleep mask 104 can be folded down into a small area to fit in a bag 306 or some other storage container. With a smaller and more compact storing arrangement, the device 100 is easier to transport and keep inside the user's bedroom 308.

As shown in FIG. 4, in one embodiment, the sleep mask 104 can be a gel sleep mask 400/402 (which can be heated or cooled, as desired), as well, to help users who might require either temperature. This can be achieved by an attachment of a sleep mask having a gel material within it that can be heated or cooled, as desired, to the hook and loop fasteners 106 on the elastic edge 202 of the bonnet component 102. This gel sleep mask 400/402 can be frozen to cool it down or heated to warm it up, thus achieving the temperature relief necessary to the user.

In a separate embodiment, the detachable sleep mask 104 can also be weighted. This weighted sleep mask 408 would still attach to the hook and loop fasteners 106 on the elastic edge 202 of the bonnet component 102 but would be heavier than a traditional sleep mask to help people with anxiety feel calmer because of an increase in pressure or weight.

In one embodiment, the detachable sleep mask 104 can be a blackout sleep mask. Further, the sleep masks 104 can include an interchangeable set of a regular mask 104, hot/cold therapy mask 400, 402, a weighted sleep mask 408, and a blackout mask 412, and could be packaged as a kit.

In one embodiment of the device 100, the detachable sleep mask 104 is either a hot sleep mask 400 or a cold sleep mask 402. These different styles of sleep masks can be used to help increase the comfortability of the user 112. In order to accomplish this style of sleep mask, the detachable sleep mask 104 must contain a pocket 404 sewn within the detachable sleep mask 104 to allow a gel insert 406 to enter. This gel insert 406 can be frozen to cool it down or heated to warm it up, allowing the detachable sleep mask 104 to be hot or cold.

In yet another embodiment, the sleep cap device 100 comprises a plurality of indicia 410. The bonnet component 102 of the device 100 may include advertising, a trademark, or other letters, designs, or characters, printed, painted, stamped, or integrated into the bonnet component 102, or any other indicia 410 as is known in the art. Specifically, any suitable indicia 410 as is known in the art can be included, such as but not limited to, patterns, logos, emblems, images, symbols, designs, letters, words, characters, animals, advertisements, brands, etc., that may or may not be bonnet, detachable sleep mask, or brand related.

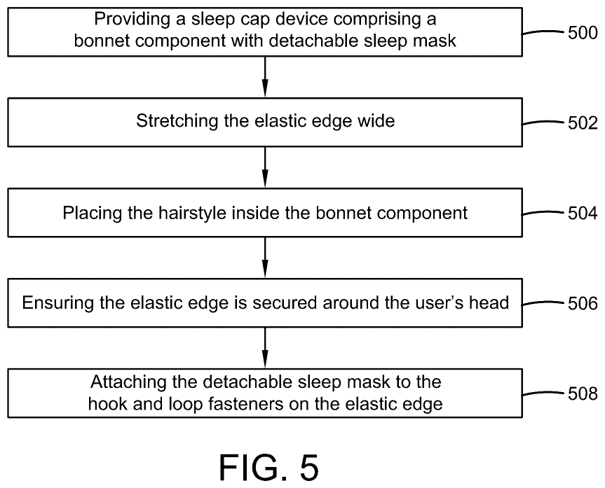

FIG. 5 illustrates a flowchart of the method of protecting a hairstyle while sleeping. The method includes the steps of at 500, providing a sleep cap device comprising a bonnet component with detachable sleep mask. The method also comprises at 502, stretching the elastic edge wide. Further, the method comprises at 504, placing the hairstyle inside the bonnet component. The method also comprises at 506, ensuring the elastic edge is secured around the user's head. Finally, the method comprises at 508, attaching the detachable sleep mask to the hook and loop fasteners on the elastic edge.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “sleep cap device”, “sleep device”, “bonnet device”, and “device” are interchangeable and refer to the sleep cap device 100 of the present invention.

Notwithstanding the forgoing, the sleep cap device 100 of the present invention can be of any suitable size and configuration as is known in the art without affecting the overall concept of the invention, provided that it accomplishes the above stated objectives. One of ordinary skill in the art will appreciate that the sleep cap device 100 as shown in FIGS. 1-5 is for illustrative purposes only, and that many other sizes and shapes of the sleep cap device 100 are well within the scope of the present disclosure. Although the dimensions of the sleep cap device 100 are important design parameters for user convenience, the sleep cap device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sleep cap device that provides a user with an improved sleep cap or bonnet and a detachable sleep mask, the sleep cap device comprising:
a bonnet component; and
detachable sleep mask; and
wherein the detachable sleep mask is secured to the bonnet component and can be attached and detached at will; and
further wherein the bonnet component and the detachable sleep mask can be collapsed and compacted for storage.

2. The sleep cap device of claim 1, wherein the bonnet component is configured in a traditional bonnet shape with an outer surface, an inner surface, and an elastic edge.

3. The sleep cap device of claim 2, wherein the elastic edge of the bonnet component contains a securing means to allow the detachable sleep mask to connect to the body component.

4. The sleep cap device of claim 3, wherein the securing means is hook and loop fasteners.

5. The sleep cap device of claim 4, wherein the bonnet component is made from silk or satin material.

6. The sleep cap device of claim 5, wherein the elastic component contains a ribbon around it to secure the bonnet component on a user's head and provide more comfort.

7. The sleep cap device of claim 6, wherein the detachable sleep mask is a gel sleep mask which can be heated or cooled, as desired.

8. The sleep cap device of claim 7, wherein the detachable sleep mask is weighted for anxiety-relief.

9. The sleep cap device of claim 8, wherein the bonnet component and detachable sleep mask can be condensed into a bag for storage in a user's bedroom.

10. The sleep cap device of claim 9 further comprising a plurality of indicia.

11. The sleep cap device of claim 10, wherein the bonnet component completely wraps around a hairstyle or head of a user and so can vary in both size and shape.

12. The sleep cap device of claim 11, wherein the detachable sleep mask is made from cotton, polyester, nylon, silk or satin material.

13. A sleep cap device that provides a user with an improved sleep cap or bonnet and a detachable sleep mask, the sleep cap device comprising:
a bonnet component configured in a traditional bonnet or sleep cap shape; and
detachable sleep mask;
wherein the bonnet component comprises an outside surface, an inside surface, and an elastic edge;
wherein the bonnet component completely covers and protects a user's head and intended hairstyle;
wherein the detachable sleep mask is secured to the elastic edge of the bonnet component so that it can be attached or detached, as needed; and
further wherein the bonnet component and the detachable sleep mask can be collapsed and compacted for storage.

14. The sleep cap device of claim 13, wherein an optional ribbon surrounds the elastic edge of the bonnet component to secure the bonnet component to the user's head and protect the user from the discomfort of hook and loop fasteners.

15. The sleep cap device of claim 14, wherein the bonnet component is comprised of a silk or satin material for protection and comfortability.

16. The sleep cap device of claim 15, wherein the detachable sleep mask is secured to the elastic edge of the bonnet component via hook and loop fasteners.

17. The sleep cap device of claim 16, wherein the detachable sleep mask is also comprised of a cotton, polyester, nylon, silk or satin material.

18. The sleep cap device of claim 13, wherein the detachable sleep mask includes a sewn-in pocket for insertion of a gel insert that is either frozen or heated.

19. The sleep cap device of claim 13, wherein the detachable sleep mask is a blackout mask.

20. A method of protecting a hairstyle while sleeping, the method comprising the following steps:
providing a sleep cap device comprising a bonnet component with detachable sleep mask;
stretching the elastic edge wide;
placing the hairstyle inside the bonnet component;
ensuring the elastic edge is secured around the user's head; and
attaching the detachable sleep mask to the hook and loop fasteners on the elastic edge.
